(19) United States
(12) Patent Application Publication
(10) Pub. No.: US 2025/0229091 A1
(43) Pub. Date: Jul. 17, 2025

(54) EXTERNAL PORTION OF MEDICAL IMPLANT WITH COMPLIANT SKIN-CONTACTING SURFACE

(71) Applicant: Cochlear Limited, Macquarie University, NSW (AU)

(72) Inventor: Harald Kreicers, Marsfield (AU)

(21) Appl. No.: 18/848,818

(22) PCT Filed: Mar. 16, 2023
(86) PCT No.: PCT/IB2023/052594

§ 371 Date: Sep. 19, 2024

Related U.S. Application Data
(60) Provisional application No. 63/363,546, filed on Apr. 25, 2022.

(51) Int. Cl. A61N 1/372; A61N 1/36; A61N 1/375

(52) U.S. Cl. CPC A61N 1/37229; A61N 1/36038; A61N 1/3758

(57) ABSTRACT

An apparatus includes a housing configured to be worn on a recipient's body, circuitry contained within the housing, the circuitry configured to be in wireless communication with an implanted device within the recipient's body, and at least one magnet contained within the housing and configured to interact with the implanted device to generate an attractive magnetic force configured to hold the housing on the recipient's body. The apparatus further includes a concave and resilient portion configured to contact the recipient's body while the housing is held by the magnetic force on the recipient's body. The portion is configured to flex in response to being pressed against the recipient's body by the magnetic force.

### BACKGROUND

### Field

The present application relates generally to systems and methods for positioning an external portion of a medical device implanted on or within a recipient's body.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a housing configured to be worn on a recipient's body. The apparatus further comprises circuitry contained within the housing. The circuitry is configured to be in wireless communication with an implanted device within the recipient's body. The apparatus further comprises at least one magnet contained within the housing. The at least one magnet is configured to interact with the implanted device to generate an attractive magnetic force configured to hold the housing on the recipient's body. The apparatus further comprises a concave and resilient portion configured to contact the recipient's body while the housing is held by the magnetic force on the recipient's body. The portion is configured to flex in response to being pressed against the recipient's body by the magnetic force.

In another aspect disclosed herein, a method comprises providing a first device configured to be worn on a recipient's skin over a second device implanted beneath the recipient's skin. The first device comprises a compliant housing portion configured to contact the recipient's skin. The method further comprises magnetically holding the first device over the second device with the compliant housing portion pressed against the recipient's skin. The method further comprises increasing a contact area of the compliant housing portion pressed against the recipient's skin.

In another aspect disclosed herein, an apparatus comprises at least one external magnet configured to be held onto a recipient's scalp by an attractive magnetic force between the at least one external magnet and at least one internal magnetic material of an implanted device beneath the recipient's scalp. The apparatus further comprises at least one external radio-frequency (RF) coil configured to be in wireless communication with at least one internal RF coil of the implanted device. The apparatus further comprises a housing portion comprising at least one flexible material configured to contact the recipient's scalp. The housing portion comprises a surface configured to substantially conform to a curvature of the recipient's scalp in response to being pressed against the recipient's scalp by the attractive magnetic force.

In another aspect disclosed herein, an apparatus comprises at least one external device configured to be held on a recipient's skin over at least one internal device beneath the recipient's skin by an attractive force between the at least one external device and the at least one internal device. The at least one external device comprises a resilient wall having a surface configured to, in response to being pressed against the recipient's skin by the attractive force, contact the recipient's skin, substantially conform to a curvature of the recipient's skin, and generate a restoring force pressing against the recipient's skin.

### BRIEF DESCRIPTION OF THE DRAWINGS

Implementations are described herein in conjunction with the accompanying drawings, in which:

FIG. 1A is a perspective view of an example cochlear implant auditory prosthesis implanted in a recipient in accordance with certain implementations described herein;

FIG. 1B is a perspective view of an example fully implantable middle ear implant auditory prosthesis implanted in a recipient in accordance with certain implementations described herein;

FIG. 2A schematically illustrates a cross-sectional view of an example transcutaneous system comprising an example apparatus spaced from the recipient's body in accordance with certain implementations described herein;

FIG. 2B schematically illustrates a cross-sectional view of the example transcutaneous system comprising the example apparatus of FIG. 2A in contact with the recipient's body in accordance with certain implementations described herein;

FIGS. 3A and 3B schematically illustrate two example apparatus in which the housing comprises the portion in accordance with certain implementations described herein; and

FIG. 4 is a flow diagram of an example method in accordance with certain implementations described herein.

### DETAILED DESCRIPTION

Certain implementations described herein provide an external portion of a medical device (e.g., an off-the-ear sound processor of an auditory prosthesis) configured to be worn in contact with the recipient's skin. The external portion has a concave surface configured to contact an outer surface of the recipient's skin and to resiliently flex in response to the attractive magnetic force holding the external portion against the skin. By flexing, the surface can conform the curvature of the recipient's body (e.g., head), thereby increasing the contact surface area to reduce the pressure for a given retention force and distributing the retention force more evenly across the contact surface area, both of which increase the recipient's comfort. This increased comfort allows the recipient to withstand a larger range of retention forces, thereby allowing overlap of the magnet strengths used to retain the external portion against the skin. In addition, by flexing, the surface can increase the translational friction between the external portion and the recipient's skin, thereby reducing the risk of unintentional dislodgement of the external portion from the recipient's skin.

The teachings detailed herein are applicable, in at least some implementations, to any type of implantable or non-implantable stimulation system or device (e.g., implantable or non-implantable auditory prosthesis device or system). Implementations can include any type of medical device that can utilize the teachings detailed herein and/or variations thereof. Furthermore, while certain implementations are described herein in the context of auditory prosthesis devices, certain other implementations are compatible in the context of other types of devices or systems (e.g., smart phones; smart speakers).

Merely for ease of description, apparatus and methods disclosed herein are primarily described with reference to an illustrative medical device, namely an implantable transducer assembly including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses. In some implementations, the teachings detailed herein and/or variations thereof can be utilized in other types of prostheses beyond auditory prostheses.

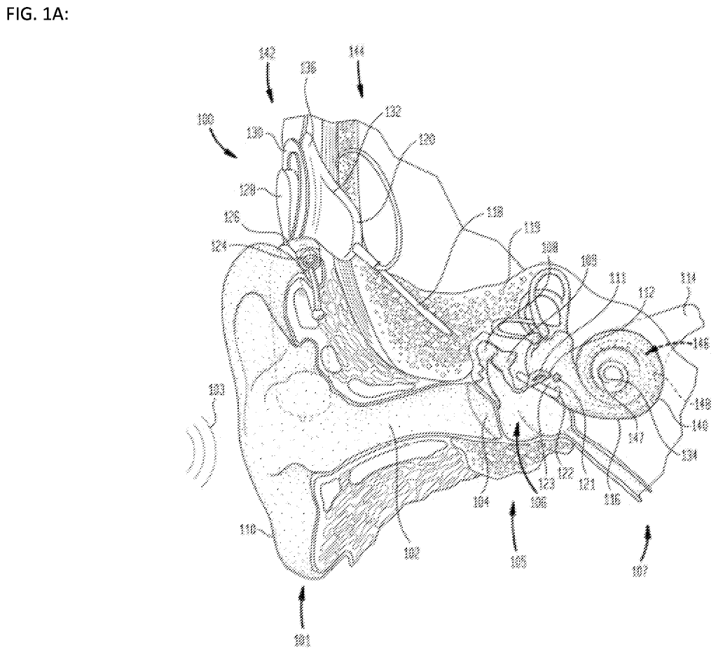

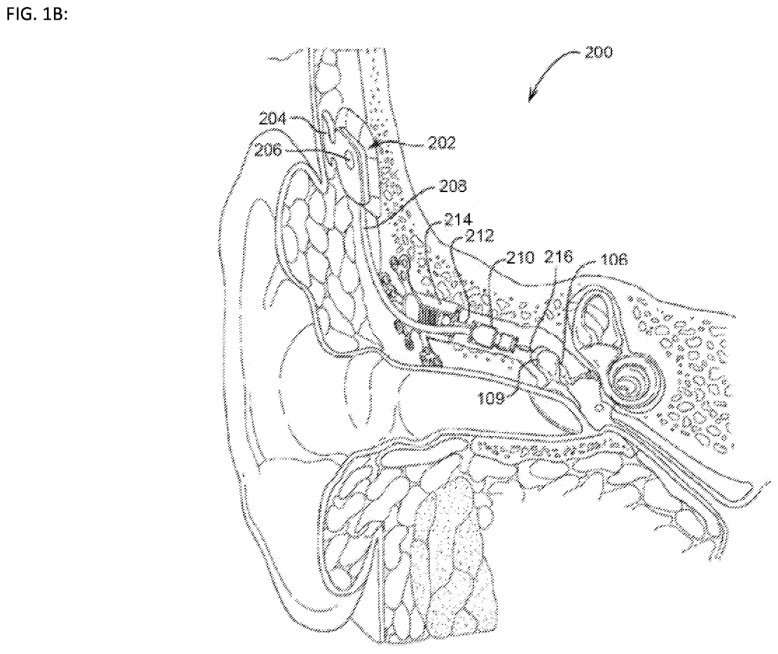

FIG. 1A is a perspective view of an example cochlear implant auditory prosthesis 100 implanted in a recipient in accordance with certain implementations described herein. The example auditory prosthesis 100 is shown in FIG. 1A as comprising an implanted stimulator unit 120 and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1A with a subcutaneously implantable microphone assembly, as described more fully herein. In certain implementations, the example cochlear implant auditory prosthesis 100 of FIG. 1A can be in conjunction with a reservoir of liquid medicament as described herein.

As shown in FIG. 1A, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1A, the example auditory prosthesis 100 comprises one or more components which are temporarily or permanently implanted in the recipient. The example auditory prosthesis 100 is shown in FIG. 1A with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1A, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1A schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

FIG. 1B schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 1B comprises a biocompatible implantable assembly 202 (e.g., comprising an implantable capsule) located subcutaneously (e.g., beneath the recipient's skin and on a recipient's skull). While FIG. 1B schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer 206 (e.g., a microphone comprising a diaphragm and an electret or piezoelectric transducer) that is positioned to receive acoustic signals through the recipient's overlying tissue. The implantable assembly 202 may further be utilized to house a number of components of the fully implantable auditory prosthesis 200. For example, the implantable assembly 202 can include an energy storage device and a signal processor (e.g., a sound processing unit). Various additional processing logic and/or circuitry components can also be included in the implantable assembly 202 as a matter of design choice.

For the example auditory prosthesis 200 shown in FIG. 1B, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). In certain implementations, the example auditory prosthesis 100, 200 shown in FIGS. 1A and 1B can comprise an implantable microphone assembly, such as the microphone assembly 206 shown in FIG. 1B. For such an example auditory prosthesis 100, the signal processor of the implantable assembly 202 can be in operative communication (e.g., electrically interconnected via a wire) with the microphone assembly 206 and the stimulator unit of the main implantable component 120. In certain implementations, at least one of the microphone assembly 206 and the signal processor (e.g., a sound processing unit) is implanted on or within the recipient.

The actuator 210 of the example auditory prosthesis 200 shown in FIG. 1B is supportably connected to a positioning system 212, which in turn, is connected to a bone anchor 214 mounted within the recipient's mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient's tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient's auditory system. To compensate for the decreased acoustic signal strength reaching the microphone assembly 202 by virtue of being implanted, the diaphragm of an implantable microphone assembly 202 can be configured to provide higher sensitivity than are external non-implantable microphone assemblies. For example, the diaphragm of an implantable microphone assembly 202 can be configured to be more robust and/or larger than diaphragms for external non-implantable microphone assemblies.

The example auditory prostheses 100 shown in FIG. 1A utilizes an external microphone 124 and the auditory prosthesis 200 shown in FIG. 1B utilizes an implantable microphone assembly 206 comprising a subcutaneously implantable acoustic transducer. In certain implementations described herein, the auditory prosthesis 100 utilizes one or more implanted microphone assemblies on or within the recipient. In certain implementations described herein, the auditory prosthesis 200 utilizes one or more microphone assemblies that are positioned external to the recipient and/or that are implanted on or within the recipient, and utilizes one or more acoustic transducers (e.g., actuator 210) that are implanted on or within the recipient. In certain implementations, an external microphone assembly can be used to supplement an implantable microphone assembly of the auditory prosthesis 100, 200. Thus, the teachings detailed herein and/or variations thereof can be utilized with any type of external or implantable microphone arrangement, and the acoustic transducers shown in FIGS. 1A and 1B are merely illustrative.

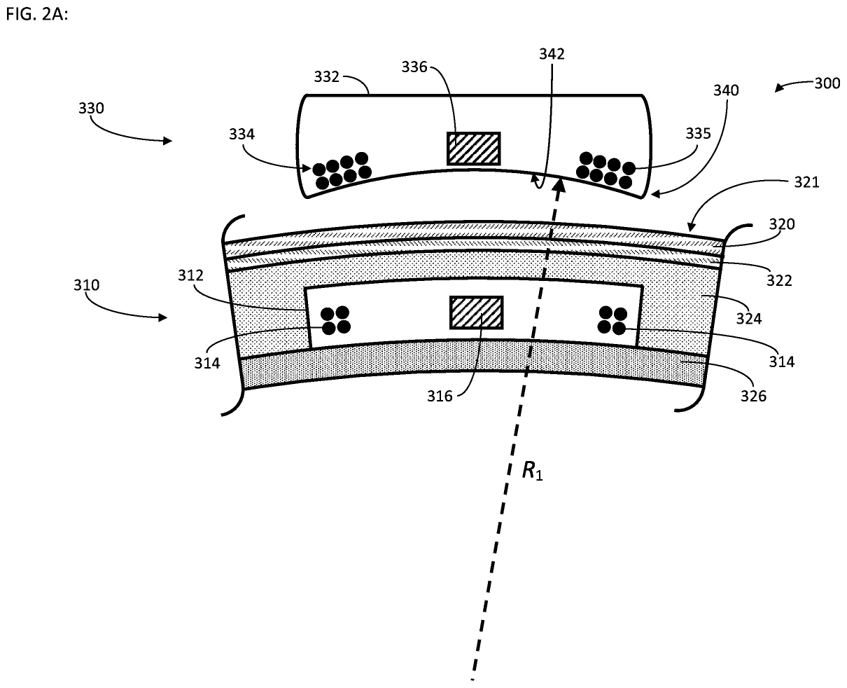

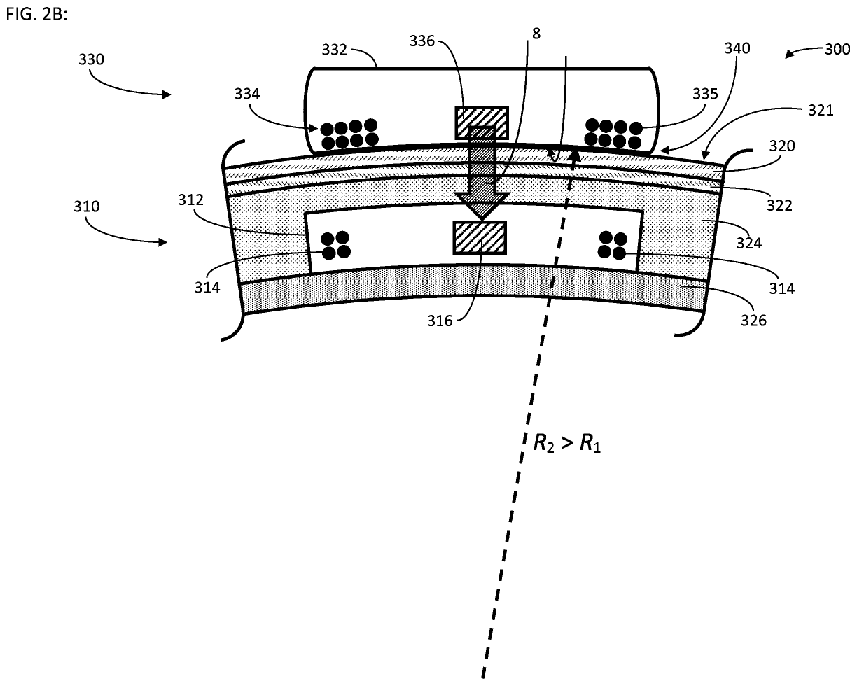

FIG. 2A schematically illustrates a cross-sectional view of an example transcutaneous system 300 comprising an example apparatus 330 spaced from the recipient's body in accordance with certain implementations described herein. FIG. 2B schematically illustrates a cross-sectional view of the example transcutaneous system 300 comprising the example apparatus 330 of FIG. 2A in contact with the recipient's body in accordance with certain implementations described herein. The example transcutaneous system 300 comprises an implantable device 310 within the recipient's body and the apparatus 330 external to the recipient's body. For example, the transcutaneous system 300 can comprise an auditory prosthesis system in which the implantable device 310 comprises one or more active elements (e.g., stimulator unit 120; assembly 202; vibrating actuator) configured to deliver stimuli to the recipient's body.

The implantable device 310 comprises at least one implantable housing 312 configured to be positioned beneath tissue of the recipient's body. For example, as shown in FIGS. 2A and 2B, the at least one implantable housing 312 is beneath the skin 320, fat 322, and/or muscular 324 layers (e.g., scalp) and above a bone 326 (e.g., skull) in a portion of the recipient's body (e.g., the head). The at least one implantable housing 312 contains at least one internal energy reception coil 314 (e.g., a substantially planar electrically conductive wire with multiple windings) and at least one internal magnetic (e.g., ferromagnetic; ferrimagnetic; permanent magnet) material 316 (e.g., disk; plate) positioned within a region at least partially bounded by the at least one internal energy reception coil 314. The at least one internal magnetic material 316 can comprise a diamagnetic magnet configured to be compatible with magnetic resonance imaging of the recipient. The at least one internal magnetic material 316 is configured to establish a magnetic attraction between the apparatus 330 and the implantable device 310 sufficient to hold the apparatus 330 against an outer surface 321 of the skin 320. The at least one implantable housing 312 can comprise a first portion configured to contain the at least one internal energy reception coil 314 and the at least one internal magnetic material 316 and a second portion configured to contain the one or more active elements, or the at least one implantable housing 312 can comprise a single housing portion configured to contain the at least one internal energy reception coil 314, the at least one internal magnetic material 316, and the one or more active elements.

In certain implementations, the apparatus 330 comprises a housing 332 configured to be worn on a recipient's body and circuitry 334 contained within the housing 332. The circuitry 334 is configured to be in wireless communication with the implanted device 310 within the recipient's body. The apparatus 330 further comprises at least one magnet 336 contained within the housing 332. The at least one magnet 336 is configured to interact with the implanted device 310 to generate an attractive magnetic force 338 configured to hold the housing 332 on the recipient's body. The apparatus 330 further comprises a concave (e.g., incurvate) and resilient (e.g., flexible; elastic) portion 340 configured to contact the recipient's body while the housing 332 is held by the magnetic force on the recipient's body. The portion 340 is configured to flex in response to being pressed against the recipient's body by the magnetic force 338.

In certain implementations, the housing 332 of the apparatus 330 is configured to be positioned on and/or over an outer surface 321 of the skin 320 and to hermetically seal the circuitry 334 and/or the at least one magnet 336 from an environment surrounding the housing 332. The housing 332 can have a width (e.g., along a lateral direction substantially parallel to the recipient's skin 320) less than or equal to 40 millimeters (e.g., in a range of 15 millimeters to 35 millimeters; in a range of 25 millimeters to 35 millimeters; in a range of less than 30 millimeters; in a range of 15 millimeters to 30 millimeters). The housing 422 can have a thickness (e.g., in a direction substantially perpendicular to the recipient's skin 320) less than or equal to 10 millimeters (e.g., in a range of less than or equal to 7 millimeters, in a range of less than or equal to 6 millimeters; in a range of less than or equal to 5 millimeters).

In certain implementations, the circuitry 334 of the apparatus 330 comprises at least one energy transmission coil 335 (e.g., a substantially planar electrically conductive wire coil with multiple windings of electrically insulated single-strand or multi-strand copper wire; copper traces on epoxy of a printed circuit board; having a substantially circular, rectangular, spiral, or oval shape or other shape). The at least one energy transmission coil 335 can have a diameter, length, and/or width (e.g., along a lateral direction substantially parallel to the recipient's skin 320) less than or equal to 40 millimeters (e.g., in a range of 15 millimeters to 35 millimeters; in a range of 25 millimeters to 35 millimeters; in a range of less than 30 millimeters; in a range of 15 millimeters to 30 millimeters). In certain implementations, the at least one energy transmission coil 335 can be sufficiently flexible to flex in response to the flexing of the portion 340, while in certain other implementations, the at least one energy transmission coil 335 is substantially rigid so as to not flex in response to the flexing of the portion 340. In certain implementations, the at least one energy transmission coil 335 is within the concave and resilient portion 340.

In certain implementations, the circuitry 334 is configured to be in wireless electrical communication (e.g., via a radio-frequency or RF link; via a magnetic induction link) with the at least one internal energy reception coil 314 when the apparatus 330 is positioned on the skin 320 of the recipient above the implanted device 310 (e.g., the apparatus 330 being held in place by the magnetic attractive force 338 between the at least one internal magnetic material 316 and the at least one magnet 336). For example, the at least one energy transmission coil 335 of the circuitry 334 can be inductively coupled with the at least one internal energy reception coil 314 and configured to wirelessly transmit electrical power to the at least one internal energy reception coil 314 and/or configured to wirelessly transmit information (e.g., data signals; control signals) to and/or to wirelessly receive information from the at least one internal energy reception coil 314.

In certain implementations, the circuitry 334 further comprises one or more microprocessors (e.g., application-specific integrated circuits; generalized integrated circuits programmed by software with computer executable instructions; microelectronic circuitry; microcontrollers) configured to control operation of the apparatus 330 and/or the implanted device 310 (e.g., set or adjust parameters of the energy transfer in response to user input and/or conditions during operation). In certain implementations, the circuitry 334 further comprises at least one storage device (e.g., at least one tangible or non-transitory computer readable storage medium; read only memory; random access memory; flash memory) in operative communication with the one or more microprocessors. The at least one storage device can be configured to store information (e.g., data; commands) accessed by the one or more microprocessors during operation. The at least one storage device can be encoded with software (e.g., a computer program downloaded as an application) comprising computer executable instructions for instructing the one or more microprocessors (e.g., executable data access logic, evaluation logic, and/or information outputting logic). In certain implementations, the one or more microprocessors execute the instructions of the software to provide functionality as described herein. In certain implementations, the circuitry 334 further comprises at least one energy storage device (e.g., battery; capacitor) configured to provide energy to the other components of the apparatus 330.

In certain implementations, the at least one magnet 336 comprises a ferromagnetic material, a ferrimagnetic material, and/or a permanent magnet (e.g., disk; plate) positioned within the housing 332. As schematically illustrated by FIGS. 2A and 2B, the at least one magnet 336 can be within a region at least partially bounded by the at least one energy transmission coil 335 of the circuitry 334 and can extend above the substantially planar energy transmission coil 335. The at least one magnet 336 can be positioned substantially centrally relative to the housing 332 and/or substantially concentrically with (e.g., centered over) the at least one energy transmission coil 335.

In certain implementations, the at least one magnet 336 is configured to establish the magnetic attractive force 338 between the apparatus 330 and the implanted device 310 (e.g., generate the magnetic attractive force 338 with the at least one internal magnetic material 316 of the implanted device 310) sufficient to hold the apparatus 330 on the recipient's body (e.g., with an outer surface 342 of the portion 340 pressed against the outer surface 321 of the skin 330). To produce a sufficiently strong magnetic attraction, the at least one magnet 336 can be positioned as close as possible to the surface 342 that contacts the recipient's skin 320, thereby minimizing the distance between the at least one magnet 336 and the at least one internal magnetic material 316. The attractive magnetic force 338 extends along a direction substantially perpendicular to the portion 340 (e.g., substantially perpendicular to the outer surface 342; substantially perpendicular to the recipient's skin 320).

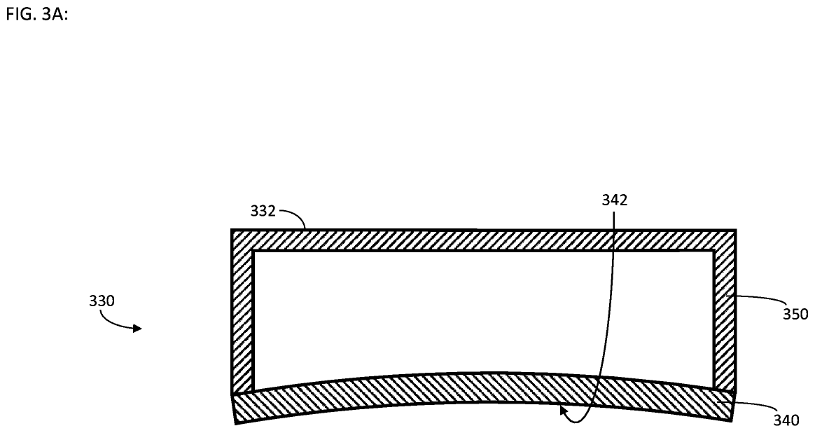

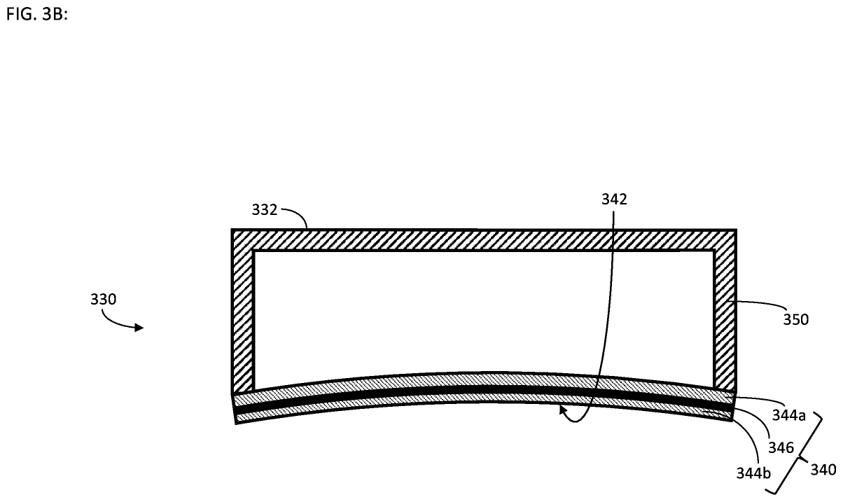

FIGS. 3A and 3B schematically illustrate two example apparatus 330 in which the housing 332 comprises the portion 340 in accordance with certain implementations described herein. For example, the housing 332 can comprise an upper portion 350 mechanically coupled to the portion 340 which comprises a lower wall of the housing 332. The portion 340 and the upper portion 350 can be hermetically sealed to one another such that both the portion 340 and the upper portion 350 bound the region containing the circuitry 334 and the at least one magnet 336. Since the portion 340 contacts the recipient's body, the portion 340 can comprise at least one biocompatible (e.g., skin-friendly) material. In addition, since the portion 340 is positioned between the circuitry 334 and the implanted device 310, the portion 340 can be substantially transmissive to electromagnetic or magnetic fields generated by the circuitry 334 and/or the at least one magnet 336 such that the housing 332 does not substantially interfere with the transmission of power via magnetic induction between the apparatus 330 and the implanted device 310 and/or the attractive magnetic force 338. Examples of biocompatible and substantially transmissive materials include but are not limited to: polymer; rubber; silicone. Since the upper portion 350 does not contact the recipient's body, the upper portion 350 can comprise at least one rigid material, as schematically illustrated by FIGS. 3A and 3B. Examples of rigid materials compatible with certain implementations described herein include but are not limited to: metals, plastics, ceramics.

In certain implementations, the portion 340 comprises an outer surface 342 configured to contact the recipient's body. The surface 342 can be configured to flex in response to being pressed against the outer surface 321 of the recipient's skin 320 (e.g., scalp) by the magnetic force 338 between the at least one magnet 336 and the at least one internal magnetic material 316. In certain implementations, as schematically illustrated by FIG. 3A, the portion 340 is substantially homogeneous and comprises at least one flexible (e.g., elastic) material. The at least one flexible material can be sufficiently resilient such that the portion 340, in response to being pressed against the recipient's body by the magnetic force 338, generates a restoring force in response to being flexed, the restoring force pressing against the recipient's body (e.g., in a direction substantially opposite to the flexing direction). Examples of flexible materials compatible with certain implementations described herein include but are not limited to: rubber; silicone.

In certain implementations, the portion 340 is substantially heterogeneous and comprises at least one non-rigid material and at least one resilient material. For example, as schematically illustrated by FIG. 3B, the portion 340 can comprise at least one non-rigid (e.g., soft) layer 344 configured to be in contact with the recipient's body and at least one resilient (e.g., flexible; elastic) layer 346 configured to stiffen the portion 340 (e.g., to generate a restoring force in response to being flexed, the restoring force in a direction substantially opposite to the flexing direction). Examples of materials for the at least one non-rigid layer 344 include but are not limited to: rubber; silicone. Examples of materials for the at least one resilient layer 346 include but are not limited to: plastic; metal. While FIG. 3B schematically illustrates an example apparatus 330 in which the at least one resilient layer 346 is sandwiched between two non-rigid layers 334a,b, other example apparatus 330 can have other configurations. For example, the at least one resilient layer 346 can be sandwiched between the upper portion 350 of the housing 332 and the at least one non-rigid layer 344.

As schematically illustrated by FIGS. 2A and 2B, the outer surface 342 of the concave and resilient portion 340 can have a first curvature when the surface 342 is not pressed against the recipient's body by the magnetic force 338 and can have a second curvature when the surface 342 is pressed against the recipient's body by the magnetic force 338. The surface 342, in response to being pressed against the recipient's body by the magnetic force 338, changes (e.g., flexes) from the first curvature to the second curvature, and, in response to be removed from the recipient's body, returns (e.g., relaxes back) to the first curvature. The first curvature can have a first radius of curvature R₁ (e.g., in the cross-sectional plane of FIGS. 2A and 2B) and the second curvature can have a second radius of curvature R₂ (e.g., in the same cross-sectional plane of FIGS. 2A and 2B), the second radius of curvature R₂ greater than the first radius of curvature R₁. As schematically illustrated in FIG. 2B, the outer surface 321 of the recipient's skin 320 beneath the apparatus 330 has the second radius of curvature R₂, and in response to being pressed against the outer surface 321 of the recipient's skin 320 by the attractive magnetic force 338, the portion 340 flexes such that the surface 342 substantially conforms to the outer surface 321 of the recipient's skin 320 (e.g., to a curvature of the recipient's scalp). As schematically illustrated in FIG. 2A, both before contacting the outer surface 321 of the recipient's skin 320 and after being removed from contacting the outer surface 321 of the recipient's skin 320, the surface 342 has a more concave curvature than when in contact with the outer surface 321 of the recipient's skin 320.

In certain implementations, the relative positions of the at least one magnet 336 and the at least one energy transmission coil 335 are configured to change in response to the flexing of the portion 340. For example, when the apparatus 330 is not in contact with the recipient's body (see, e.g., FIG. 2A), the at least one magnet 336 is higher relative to the at least one energy transmission coil 335 than when the apparatus 330 is in contact with the recipient's body (see, e.g., FIG. 2B). The flexing of the portion 340 in certain implementations allows the at least one energy transmission coil 335 to be positioned closer to the at least one internal energy reception coil 314 than if the portion 340 were inflexible, thereby improving the coupling coefficient (e.g., maximizing the power transfer efficiency and/or the inductive coupling) between the at least one energy transmission coil 335 and the at least one internal energy reception coil 314. In contrast to other systems which add a soft pad or spacer to a rigid lower surface of the housing 332, the flexible surface 340 of certain implementations described herein does not increase a distance between the at least one magnet 336 and the at least one internal magnetic material 316 or a distance between the at least one energy transmission coil 335 and the at least one internal energy reception coil 314.

In certain implementations, the portion 340 allows the housing 332 to conform to the curvature of the recipient's body (e.g., head), thereby increasing (e.g., maximizing) the surface area of the portion 340 contacting the recipient's skin 320 and reducing (e.g., minimizing) the pressure applied to the recipient's skin 320 by the apparatus 330 for a given magnetic force 338, thereby increasing the comfort to the recipient when wearing the apparatus 330. For example, substantially all of the surface area of the outer surface 342 can be in contact with the outer surface 321 of the recipient's skin 320, thereby distributing the magnetic attractive force 338 substantially evenly across substantially all of the surface area of the outer surface 342. In addition, the increased surface area can increase (e.g., maximize) the translational friction between the outer surface 321 of the recipient's skin 320 and the outer surface 342 of the portion 340, thereby reducing (e.g., minimizing) the risk of the apparatus 330 being removed (e.g., dislodged; knocked off) from the recipient's body unintentionally.

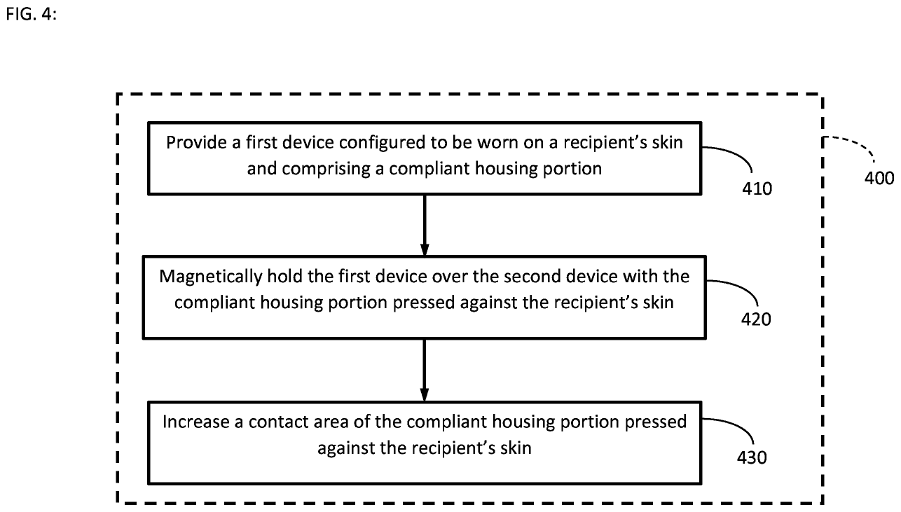

FIG. 4 is a flow diagram of an example method 400 in accordance with certain implementations described herein. While the method 400 is described by referring to some of the structures of the example apparatus 300 of FIGS. 2A-2B and 3A-3B, other apparatus and systems with other configurations of components can also be used to perform the method 400 in accordance with certain implementations described herein.

In an operational block 410, the method 400 comprises providing a first device (e.g., apparatus 300) configured to be worn on a recipient's skin 320 over a second device (e.g., implanted device 310) implanted beneath the recipient's skin 320. The first device comprises a compliant housing portion (e.g., portion 340) configured to contact the recipient's skin 320.

In an operational block 420, the method 400 further comprises magnetically holding the first device over the second device with the compliant housing portion pressed against the recipient's skin 320. For example, the first device can apply a pressure to the recipient's skin 320 by pressing the compliant housing portion against the recipient's skin 320. In certain implementations, magnetically holding the first device over the second device comprises generating an attractive magnetic force (e.g., force 338) between the first device and the second device. The attractive magnetic force can extend along a direction substantially perpendicular to an outer surface (e.g., surface 342) of the compliant housing portion and substantially concentric with the outer surface.

In an operational block 430, the method 400 further comprises increasing a contact area of the compliant housing portion pressed against the recipient's skin 320. For example, increasing the contact area can comprise reducing the pressure applied to the recipient's skin 320 by the first device while increasing a translational friction force between the compliant housing portion and the recipient's skin 320.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of various devices, various implementations described herein can be incorporated in a variety of other suitable devices, methods, and contexts. More generally, as can be appreciated, certain implementations described herein can be used in a variety of implantable medical device contexts that can benefit from certain attributes described herein.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein but should be defined only in accordance with the claims and their equivalents.

1. An apparatus comprising:
a housing configured to be worn on a recipient's body;
circuitry contained within the housing, the circuitry configured to be in wireless communication with an implanted device within the recipient's body;
at least one magnet contained within the housing, the at least one magnet configured to interact with the implanted device to generate an attractive magnetic force configured to hold the housing on the recipient's body; and
a concave and resilient portion configured to contact the recipient's body while the housing is held by the magnetic force on the recipient's body, the portion configured to flex in response to being pressed against the recipient's body by the magnetic force.

2. The apparatus of claim 1, wherein the circuitry comprises a substantially planar wire coil.

3. The apparatus of claim 2, wherein the at least one magnet extends above the substantially planar wire coil.

4. The apparatus of claim 3, wherein the at least one magnet is centered over the substantially planar wire coil.

5. The apparatus of claim 1, wherein the portion comprises a surface configured to contact the recipient's body and the magnetic force extends along a direction substantially perpendicular to the surface.

6. The apparatus of claim 5, wherein the surface has a first curvature when the surface is not pressed against the recipient's body by the magnetic force and has a second curvature when the surface is pressed against the recipient's body by the magnetic force.

7. The apparatus of claim 6, wherein the surface, in response to being pressed against the recipient's body by the magnetic force, changes from the first curvature to the second curvature, and, in response to being removed from the recipient's body, returns to the first curvature.

8. The apparatus of claim 6, wherein the first curvature has a first radius and the second curvature has a second radius, the second radius larger than the first radius.

9. The apparatus of claim 1, wherein the portion, in response to being pressed against the recipient's body by the magnetic force, generates a restoring force that presses against the recipient's body.

10. The apparatus of claim 1, wherein the portion has a surface area contacting the recipient's body and the magnetic force generates a pressure against the recipient's body that is distributed substantially equally across the entire surface area.

11. The apparatus of claim 1, wherein the at least one magnet and at least a portion of the circuitry are configured to move relative to one another in response to flexing of the portion.

12. The apparatus of claim 1, wherein the portion comprises at least one non-rigid layer comprising rubber and/or silicone and at least one resilient layer comprising plastic and/or metal.

13. A method comprising:
providing a first device configured to be worn on a recipient's skin over a second device implanted beneath the recipient's skin, the first device comprising a compliant housing portion configured to contact the recipient's skin;
magnetically holding the first device over the second device with the compliant housing portion pressed against the recipient's skin; and
increasing a contact area of the compliant housing portion pressed against the recipient's skin.

14. The method of claim 13, wherein said magnetically holding the first device over the second device comprises applying a pressure to the recipient's skin by pressing the compliant housing portion against the recipient's skin.

15. The method of claim 14, wherein said increasing the contact area comprises reducing the pressure while increasing a translational friction force between the compliant housing portion and the recipient's skin.

16. The method of claim 13, wherein said magnetically holding the first device over the second device comprises generating an attractive magnetic force between the first device and the second device, the attractive magnetic force extending along a direction substantially perpendicular to an outer surface of the compliant housing portion and substantially concentric with the outer surface.

17. An apparatus comprising:
at least one external magnet configured to be held onto a recipient's scalp by an attractive magnetic force between the at least one external magnet and at least one internal magnetic material of an implanted device beneath the recipient's scalp;
at least one external radio-frequency (RF) coil configured to be in wireless communication with at least one internal RF coil of the implanted device; and
a housing portion comprising at least one flexible material configured to contact the recipient's scalp, the housing portion comprising a surface configured to substantially conform to a curvature of the recipient's scalp in response to being pressed against the recipient's scalp by the attractive magnetic force.

18. The apparatus of claim 17, wherein the surface is configured to flex such that the at least one external magnet is closer to the at least one internal magnetic material.

19. The apparatus of claim 17, wherein said flexing of the surface increases a coupling coefficient between the at least one external RF coil and the at least one internal RF coil.

20. The apparatus of claim 17, wherein said flexing of the surface increases an area of the surface in contact with the recipient's scalp.

21. The apparatus of claim 17, wherein the at least one external magnet and the at least one external RF coil are configured to move relative to one another in response to flexing of the surface.

22. An apparatus comprising:
at least one external device configured to be held on a recipient's skin over at least one internal device beneath the recipient's skin by an attractive force between the at least one external device and the at least one internal device, the at least one external device comprising a resilient wall having a surface configured to, in response to being pressed against the recipient's skin by the attractive force, contact the recipient's skin, substantially conform to a curvature of the recipient's skin, and generate a restoring force pressing against the recipient's skin.

23. The system of claim 22, wherein the at least one external device comprises circuitry configured to be in wireless and transcutaneous communication with the at least one internal device.

24. The system of claim 22, wherein the at least one external device comprises at least one magnet and the at least one internal device comprises at least one ferromagnetic material, the attractive force generated by the at least one magnet and the at least one ferromagnetic material.

25. The system of claim 22, wherein the surface is concave and has a first curvature prior to being pressed against the recipient's skin by the attractive force, a second curvature while pressed against the recipient's skin by the attractive force, and the first curvature after being removed from the recipient's skin.

26. The apparatus of claim 25, wherein the first curvature has a first radius and the second curvature has a second radius, the second radius larger than the first radius.

27. The apparatus of claim 22, wherein the at least one external device comprises a sound processor of an auditory prosthesis and the at least one internal device comprises a stimulator unit of the auditory prosthesis, the stimulator unit configured to apply stimulation signals comprising auditory information to the recipient's body.
